(19) United States
(12) Patent Application Publication
(10) Pub. No.: US 2010/0216245 A1
(43) Pub. Date: Aug. 26, 2010

(54) FROZEN CELL IMMOBILIZED PRODUCT, PRIMARY HEPATOCYTE CULTURE TOOL, AND METHOD FOR PRODUCING PRIMARY HEPATOCYTE CULTURE TOOL

(75) Inventors: Shin Enosawa, Tokyo (JP); Yoshitaka Miyamoto, Aichi (JP); Takeshi Ikeya, Chiba (JP)

Correspondence Address: YOUNG & THOMPSON, 209 Madison Street, Suite 500, Alexandria, VA, 22314, US

(73) Assignees: NATIONAL CENTER FOR CHILD HEALTH AND DEVELOPMENT, Tokyo (JP); TOYO GOSEI CO., LTD., Ichikawa-shi, Chiba (JP)

(21) Appl. No.: 12/738,809

(22) PCT Filed: Sep. 11, 2008
(86) PCT No.: PCT/JP2008/066438

§ 371 Date: Apr. 19, 2010

(30) Foreign Application Priority Data
Oct. 19, 2007 (JP) 2007-273099

(51) Int. Cl. C12N 5/07

(52) U.S. Cl. 435/402

(57) ABSTRACT

The present invention provides a frozen cell immobilized product which is obtained by applying a technique for freezing cultured cells to a technique for forming primary hepatocyte spheroids through co-culturing, and which can improve performance in an examination or a test using the technique for forming primary hepatocyte spheroids; a primary hepatocyte culture tool; and a method for producing the primary hepatocyte culture tool. According to the invention, a cell-adhesion region of a culture substrate is defined in a pattern; animal-derived adherent cells are cultured on the cell-adhesion region; and the cultured cells are frozen together with a freezing culture medium.

### TECHNICAL FIELD

The present invention relates to a frozen cell immobilized product prepared by culturing animal-derived adherent cells on a substrate in a pattern, followed by freezing. The present invention also relates to a primary hepatocyte culture tool obtained by thawing the frozen cell immobilized product and seeding primary hepatocytes onto the product within a predetermined period of time after thawing. The present invention also relates to a method for producing the primary hepatocyte culture tool.

### BACKGROUND ART

Hitherto, several techniques have been proposed for culturing cells in a pattern. For example, there has been proposed a patterned culture substrate product which is produced by applying an aqueous photosensitive material on a substrate, and forming a pattern on the substrate through photolithography, wherein, upon cell culture, cells adhere to only a portion at which the original substrate is exposed (see Patent Document 1). This patterned culture substrate product realizes, for example, effective arrangement of animal-derived adherent cells in a specific region; for example, in a circular pattern of 10 μmφ to 1 mmφ. However, difficulty is encountered in transporting this culture substrate product together with a liquid culture medium while cells are caused to adhere to the substrate product, and in culturing cells on the substrate product for a long period of time.

Also, there has been proposed a technique for forming primary hepatocyte spheroids through co-culturing, which technique effectively uses cells adhering to a substrate in a pattern (see Patent Document 2). In this technique, animal-derived adherent cells are cultured as feeder cells in a circular region of about 100 μmφ, and then seeding of primary hepatocytes is carried out, to thereby form hepatocyte spheroids. This technique readily realizes long-term survival of primary hepatocytes, which has been difficult to attain. However, this technique requires a process in which preculture is carried out for 24 hours or longer after seeding of feeder cells, followed by seeding of hepatocytes, since stable hepatocyte spheroids fail to be formed without performing the process. Therefore, this technique poses a problem in terms of poor working efficiency in an examination or test using the thus-formed hepatocyte spheroids.

Meanwhile, there has been proposed a technique in which anchorage-dependent animal cells are cultured on a culture substrate; the original culture medium is replaced with a freezing culture medium; and anchorage-dependent animal cells adhering to the culture substrate are frozen together with the freezing culture medium and the culture substrate (see Patent Document 3). However, this technique is based on a simple process in which culturing of anchorage-dependent animal cells is terminated by carrying out freezing in a step of culturing the cells, and then culturing of the cells is continued after thawing. Thus, application of this technique to the aforementioned complicated system is not suggested.

Patent Document 1: Japanese Patent Application Laid-Open (kokai) No. 2005-280076 (e.g., section [0049] or [0094])

### Patent Document 2: WO 2003-10302 (e.g., “Disclosure of the Invention”)

Patent Document 3: Japanese Patent Publication (kokoku) No. H05-77389.

### DISCLOSURE OF THE INVENTION

### Problems to be Solved by the Invention

In view of the foregoing, an object of the present invention is to provide a frozen cell immobilized product which is obtained by applying a technique for freezing cultured cells to the aforementioned technique for forming primary hepatocyte spheroids through co-culturing, and which can improve performance in an examination or a test using the technique for forming primary hepatocyte spheroids. Another object of the present invention is to provide a primary hepatocyte culture tool. Yet another object of the present invention is to provide a method for producing the primary hepatocyte culture tool.

### Means for Solving the Problems

In a first mode of the present invention attaining the aforementioned objects, there is provided a frozen cell immobilized product, characterized in that the cell product comprises a culture substrate having a cell-adhesion region defined in a pattern; animal-derived adherent cells cultured on the cell-adhesion region; and a freezing culture medium; and that the cultured cells are frozen together with the freezing culture medium.

In a second mode of the present invention, there is provided a primary hepatocyte culture tool (i.e., apparatus for culturing primary hepatocytes), characterized in that the tool is produced by thawing the frozen cell immobilized product as recited in the first mode, replacing the freezing culture medium with an ordinary culture medium, and seeding primary hepatocytes on the product.

A third mode of the present invention is drawn to a specific embodiment of the primary hepatocyte culture tool according to the second mode, wherein thawing is carried out at 30 to 48° C.

In a fourth mode of the present invention, there is provided a method for producing a primary hepatocyte culture tool, characterized in that the method comprises thawing the frozen cell immobilized product as recited in the first mode; replacing the freezing culture medium with an ordinary culture medium; and seeding primary hepatocytes on the product.

A fifth mode of the present invention is drawn to a specific embodiment of the method for producing a primary hepatocyte culture tool according to the fourth mode, wherein thawing is carried out at 30 to 48° C.

A sixth mode of the present invention is drawn to a specific embodiment of the method for producing a primary hepatocyte culture tool according to the fourth or fifth mode, wherein seeding of primary hepatocytes is carried out within 24 hours after thawing.

A seventh mode of the present invention is drawn to a specific embodiment of the method for producing a primary hepatocyte culture tool according to the fourth or fifth mode, wherein seeding of primary hepatocytes is carried out within three hours after thawing.

### EFFECTS OF THE INVENTION

According to the present invention, there can be provided a frozen cell immobilized product which is obtained by culturing animal-derived adherent cells on a culture medium in a pattern, and replacing the culture medium with a freezing culture medium, followed by cryopreservation, and which can be transported or preserved for a long period of time. Through use of the frozen cell immobilized product, animal-derived adherent cells can be frozen in a pattern and preserved under frozen conditions for one year or longer without alteration of, for example, properties of the cells. When the frozen cell immobilized product is packaged together with dry ice serving as a freezing agent, the product can be transported under frozen conditions. In addition, the frozen cell immobilized product is convenient and suitable for use in, for example, preservation/transportation of cells or in screening of drugs, since separately provided primary hepatocytes can be seeded onto the product immediately after thawing thereof.

The primary hepatocyte culture tool of the present invention, which is prepared by seeding hepatocytes onto the frozen cell immobilized product immediately after thawing of the product, realizes, for example, highly effective screening of drugs; i.e., compounds which are possible drug candidates.

According to the method for producing the primary hepatocyte culture tool by use of the frozen cell immobilized product of the present invention, hepatocytes can be seeded onto the frozen cell immobilized product immediately after thawing of the product, and, for example, highly effective screening of drugs can be carried out.

### BRIEF DESCRIPTION OF THE DRAWINGS

FIG. 1 is a photograph showing the results of Example 2.

FIG. 2 is a photograph showing the results of Example 3.

### BEST MODES FOR CARRYING OUT THE INVENTION

No particular limitation is imposed on the culture substrate which may be employed in the present invention, so long as the substrate has a cell-adhesion region defined in a pattern (hereinafter may be referred to as a “patterned cell-adhesion region”). However, in the culture substrate, preferably, a region to which cells do not adhere (hereinafter the region may be referred to as a “non-cell-adhesion region”) is formed of a hydrophilic crosslinked polymer, and the cell-adhesion region (i.e., a region formed through removal of the crosslinked polymer) is defined in the non-cell-adhesion region. The crosslinked polymer forming the non-cell-adhesion region is preferably formed through photocrosslinking.

No particular limitation is imposed on the hydrophilic polymer forming such a non-cell-adhesion region. However, the hydrophilic polymer is preferably, for example, saponified polyvinyl acetate, polyethylene glycol, or polyhydroxyethyl(meth)acrylate. Of these, saponified polyvinyl acetate or polyethylene glycol is more preferred.

Photocrosslinking for forming such a crosslinked polymer readily proceeds through irradiation with, for example, UV light having a wavelength which activates a photosensitive group. Photocrosslinking is advantageous in that a patterned crosslinked polymer is readily formed by means of, for example, a light-shielding mask having a pattern. No particular limitation is imposed on the reaction for forming such a photocrosslinked polymer, and the polymer may be formed through, for example, photopolymerization employing a photopolymerization initiator, photodimerization reaction of stilbene or the like, or crosslinking reaction by photocleavage of an azido group. Of these, photocleavage reaction of an azido group is preferably employed.

No particular limitation is imposed on the pattern form defined by the non-cell-adhesion region formed by a hydrophilic crosslinked polymer and the cell-adhesion region patterned through removal of the polymer, so long as the pattern form corresponds to the intended use of the product. When, for example, the product is employed as a tool for forming primary hepatocyte spheroids, preferably, the cell-adhesion region has a hole form of 100 μmφ.

No particular limitation is imposed on the substrate on which such a patterned crosslinked polymer is provided, so long as animal-derived adherent cells of interest can adhere to the substrate. The substrate employed may be made of, for example, glass, polystyrene, polyethylene, or polypropylene, for tissue culture. Particularly, glass or polystyrene for tissue culture is preferably employed.

A substance for promoting cell adhesion may be immobilized on such a substrate. Examples of the substance for promoting cell adhesion include collagen, fibronectin, vitronectin, and poly-L-lysine.

No particular limitation is imposed on the form of the substrate employed, and the substrate may be in the form of, for example, a flat plate, a petri dish, or a multi-well plate for cell culture.

Animal-derived adherent cells are seeded on the thus-prepared culture substrate having a patterned cell-adhesion region. No particular limitation is imposed on the cells seeded, so long as the cells are animal-derived adherent cells. However, the cells are preferably vascular endothelial cells or fibroblasts. When cells are seeded onto the culture substrate having a patterned cell-adhesion region, the cells are arranged in the pattern without any particular operation after seeding.

The cell concentration of a cell suspension seeded is preferably such a level that cells are confluent in the cell-adhesion region. In the case of proliferative cells, no particular problem arises even when the cell concentration is reduced to about ⅛ the aforementioned preferred level. Even when the cell concentration is higher than that at which cell confluence is achieved, no particular problem arises, so long as cells which have not been adhere to the cell-adhesion region can be removed upon culture medium replacement. However, such a high cell concentration should be avoided, from the viewpoint of prevention of waste of cells.

After seeding of the aforementioned animal-derived adherent cells, culturing must be carried out by use of a culture medium until the cells firmly adhere to the cell-adhesion region. No particular limitation is imposed on the culturing time, so long as the cells can precipitate and adhere to the cell-adhesion region. The culturing time is, for example, 3 hours to 48 hours, preferably 24 hours to 48 hours, more preferably 24 hours.

The culture medium may be a commonly used culture medium, selected depending on the type of cells seeded. When, for example, vascular endothelial cells or fibroblasts are seeded, preferably, a 10% (V/V) fetal bovine serum-containing Dulbecco's modified Eagle's medium is employed.

After culturing for a predetermined period of time, the culture substrate having the patterned cell-adhesion region to which the cells are adhering is frozen, to thereby yield a frozen cell immobilized product. Before freezing, the original culture medium must be replaced with a freezing culture medium. This is because, when the cells are frozen while being held in a commonly used culture medium, the survival rate of the cells is considerably reduced during thawing. The freezing culture medium employed may be an appropriate culture medium containing a freezing damage preventive agent (e.g., 10% (V/V) dimethyl sulfoxide (DMSO)), and is preferably a commercially available one, such as Cellbanker (product of Juji Field Inc.) or Bambanker (product of Lymphotec Inc.). Also, addition of a sugar (e.g., a monosaccharide, a disaccharide, an oligosaccharide, or a polysaccharide) is effective for preventing freezing damage. Examples of the sugar for preventing freezing damage include glucose, lactose, trehalose, and raffinose.

Immediately after replacement with a freezing medium (e.g., within 30 minutes after replacement), freezing is carried out by means of, for example, a program freezer. No particular limitation is imposed on the freezing temperature, so long as, for example, the cells are not denatured or the survival rate of the cells is not considerably reduced upon thawing. However, the freezing temperature is preferably −20° C. to −80° C., particularly preferably −80° C.

When the culture substrate having the patterned cell-adhesion region to which the cells are adhering is frozen, generally, the substrate is placed in a container (a hermetic or non-hermetic container) in consideration of, for example, workability.

Completion of freezing yields a frozen cell immobilized product. When, for example, the frozen cell immobilized product is preserved at −80° C., the culture medium is solidified, and thus transportation of the product, which is difficult to perform at ambient temperature, can be readily carried out. When the product is transported, preferably, the product is placed in a heat-insulating container together with dry ice. When the product is in direct contact with dry ice, the product may be damaged. Therefore, preferably, the product is packaged into a bag (e.g., polyethylene bag).

Upon use of the frozen cell immobilized product, preferably, the product is thawed at 30° C. to 48° C.; for example, the product is thawed in an incubator or the like at 37° C. The freezing culture medium melts about 10 minutes after placement of the product in an incubator. Thereafter, the freezing culture medium is replaced with an ordinary culture medium employed, and then culturing is further carried out. In the case of the frozen cell immobilized product of the present invention, after thawing, the cells are not removed from the product, and the survival rate of the cells is not reduced. Therefore, the thus-thawed product can be employed as in the case of a non-frozen product.

When the thus-thawed product is employed as a primary hepatocyte culture tool, primary hepatocytes are seeded onto the product after culturing for a predetermined period of time following replacement of the freezing culture medium with an ordinary culture medium. In general, after animal-derived adherent cells have been seeded onto an ordinary culture substrate, culturing must be carried out for 24 hours or longer before seeding of primary hepatocytes, since reliable adhesion of the animal-derived adherent cells requires such a long period of time. In contrast, in the case of the frozen cell immobilized product of the present invention, animal-derived adherent cells reliably adhere to the product before freezing, and therefore hepatocytes can be seeded onto the product within a short period of time after freezing-thawing (preferably within 24 hours, more preferably within three hours after freezing-thawing). Thus, spheroids maintaining hepatocytic functions can be suitably formed. According to the primary hepatocyte culture tool or the production method therefor using the frozen cell immobilized product of the present invention, primary hepatocytes can be seeded onto the frozen cell immobilized product immediately after thawing of the product without culturing of animal cells over a long period of time (i.e., 24 hours or longer). Therefore, for example, screening of drugs can be effectively carried out.

### EXAMPLES

The present invention will next be described by way of examples, which should not be construed as limiting the invention thereto.

### Example 1

### Production of Frozen Cell Immobilized Product (Preparation of a Culture Substrate Having a Patterned Cell-Adhesion Region)

A water-soluble photosensitive material mainly containing saponified polyvinyl acetate was prepared in a manner similar to that described in Examples of Japanese Patent Application Laid-Open (kokai) No. 2005-280076, and a water-soluble photosensitive material mainly containing polyethylene glycol was prepared in a manner similar to that described in Examples of Japanese Patent Application Laid-Open (kokai) No. 2006-307184. Each of these photosensitive materials was applied to a glass thin plate of 21 mmφ (product of Matsunami Glass Ind., Ltd.) and a 12-well plate (product of Sumitomo Bakelite Co., Ltd.), followed by light exposure and development through a mask having a pattern, to thereby yield a patterned glass thin plate for culture and a patterned 12-well plate for culture, each having numerous holes (corresponding to exposed portions of the substrate) of 100 μmφ (2,500 holes/cm²) (i.e., numerous patterned cell-adhesion regions).

(Preparation of Frozen Cell Immobilized Product)

Among the thus-obtained substrates, the patterned glass thin plate for culture was placed on the bottom of each well of a 12-well plate (product of Sumitomo Bakelite Co., Ltd.), and then was sterilized through irradiation with UV light. Separately, the patterned 12-well plate for culture was sterilized through irradiation with UV light. Subsequently, a cell suspension prepared by suspending bovine vascular endothelial cells or mouse fibroblasts in a 10% (V/V) fetal bovine serum-containing Dulbecco's modified Eagle's medium (cell concentration: 2×10⁵ cells/mL) was added to the plate (1 mL/well), followed by culturing in a CO₂ incubator (CO₂ concentration: 5%) at 37° C. for 24 hours or 48 hours.

As described above, in each of the culture substrates, the original culture medium was replaced with a freezing culture medium (0.5 mL). The below-described nine types of freezing culture media (1) to (9) were employed.

Immediately after culture medium replacement, each substrate was frozen in a freezer at −20° C. or −80° C., to thereby yield a frozen cell immobilized product.

(1) 10% (V/V) Fetal bovine serum-containing Dulbecco's modified Eagle's medium to which 10% (V/V) dimethyl sulfoxide had been added
(2) 10% (V/V) Fetal bovine serum-containing Dulbecco's modified Eagle's medium to which 15% (V/V) dimethyl sulfoxide had been added
(3) (1)+0.05M (mol/L) Trehalose

### (4) (1)+0.1M Trehalose

### (5) (1)+0.2M Trehalose

### (6) (1)+0.05M Raffinose

### (7) (1)+0.1M Raffinose

### (8) (1)+0.2M Raffinose

(9) Cellbanker (commercial product)

### Example 2

### Evaluation of Performance of Frozen Cell Immobilized Product, Production of Primary Hepatocyte Culture Tool, and Working of Production Method of Primary Hepatocyte Culture Tool

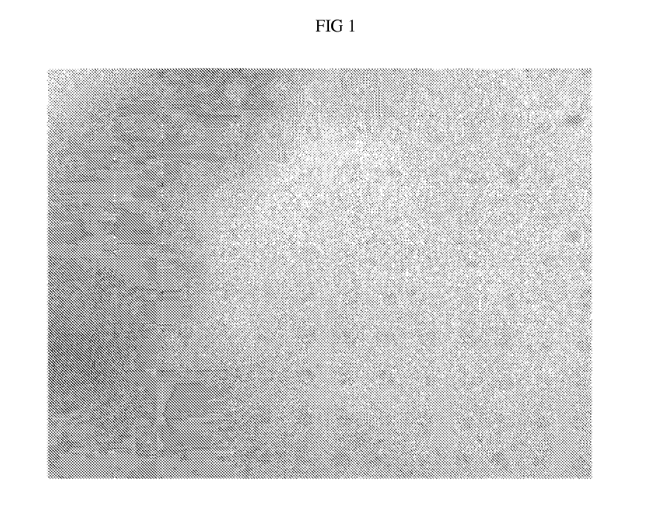

For evaluation of the performance of each of the frozen cell immobilized products prepared in Example 1, the shape of cells after thawing was observed, and the survival rate and growth rate of cells removed from the product were determined. Frozen cell immobilized products cryopreserved for two months, one month, or one week were evaluated. After completion of cryopreservation, an ordinary culture medium (1 mL) was added to each product, and the product was allowed to stand still in an incubator at 37° C. for 10 minutes, followed by replacement of the culture medium with an ordinary 10% (V/V) fetal bovine serum-containing Dulbecco's modified Eagle's medium. While culturing was performed in the thus-replaced medium, the shape of cells was observed under a microscope 3 hours or 24 hours after culture medium replacement. For example, FIG. 1 shows the results obtained through microscopic observation 3 hours after culture medium replacement. Other conditions are as follows: water-soluble photosensitive material: material mainly containing polyethylene glycol; substrate: patterned glass thin plate for culture; animal-derived adherent cells: bovine vascular endothelial cells; preculture: 24 hours; freezing culture medium: Cellbanker; and cryopreservation period: two months).

Unlike the case of preservation at −80° C., in the case of preservation at −20° C., slight removal of cells from the product was observed 3 hours or 24 hours after culture medium replacement. However, the degree of cell removal was found to be such a level that no problem arises upon use of the product. Thereafter, the product was washed with phosphate buffer, and then cells were recovered from the product by use of a trypsin solution, followed by trypan blue staining of a portion of the recovered cells, to thereby determine the survival rate of the cells. As a result, the survival rate was found to be 99% or higher under all the experimental conditions.

For evaluation of cell growth potential, cells were recovered from each of the frozen cell immobilized products prepared by use of freezing media shown in Tables 1 and 2 (preparation conditions are as follows: water-soluble photosensitive material: material mainly containing polyethylene glycol; substrate: patterned glass thin plate for culture; animal-derived adherent cells: bovine vascular endothelial cells; preculture: 24 hours; and cryopreservation period: one month), and the thus-recovered cells were further cultured in a culture flask. As a result, cell growth was observed under all the experimental conditions, and no great variation in doubling time was observed.

TABLE 1
Evaluation results of growth of cells
recovered 3 hours after thawing

| | Cell concentration | Cell concentration | |
|---|---|---|---|
| | immediately after | after 24-hour | Growth |
| | recovery | culturing | rate |
| | (cells/mL) | (cells/mL) | (%) |
| DMSO 10% (V/V) | 0.43 × 10⁵ | 0.75 × 10⁵ | 174 |
| (no addition of sugar) | | | |
| Trehalose 0.05 M | 0.43 × 10⁵ | 0.95 × 10⁵ | 221 |
| Trehalose 0.1 M | 0.48 × 10⁵ | 1.03 × 10⁵ | 215 |
| Trehalose 0.2 M | 0.55 × 10⁵ | 1.05 × 10⁵ | 191 |
| Raffinose 0.05 M | 0.45 × 10⁵ | 0.80 × 10⁵ | 178 |
| Raffinose 0.1 M | 0.53 × 10⁵ | 0.78 × 10⁵ | 147 |
| Raffinose 0.2 M | 0.70 × 10⁵ | 1.08 × 10⁵ | 154 |
| Cellbanker | 0.75 × 10⁵ | 1.05 × 10⁵ | 140 |

TABLE 2
Evaluation results of growth of cells
recovered 24 hours after thawing

| | Cell concentration | Cell concentration | |
|---|---|---|---|
| | immediately after | after 24-hour | Growth |
| | recovery | culturing | rate |
| | (cells/mL) | (cells/mL) | (%) |
| DMSO 10% (V/V) | 0.68 × 10⁵ | 1.08 × 10⁵ | 159 |
| (no addition of sugar) | | | |
| Trehalose 0.1 M | 0.60 × 10⁵ | 1.00 × 10⁵ | 167 |
| Raffinose 0.1 M | 0.58 × 10⁵ | 0.95 × 10⁵ | 164 |
| Cellbanker | 0.63 × 10⁵ | 1.00 × 10⁵ | 159 |

### Example 3

### Transportation Test of Frozen Cell Immobilized Product, Subsequent Primary Hepatocyte Culture Tool, and Working of Production of Primary Hepatocyte Culture Tool

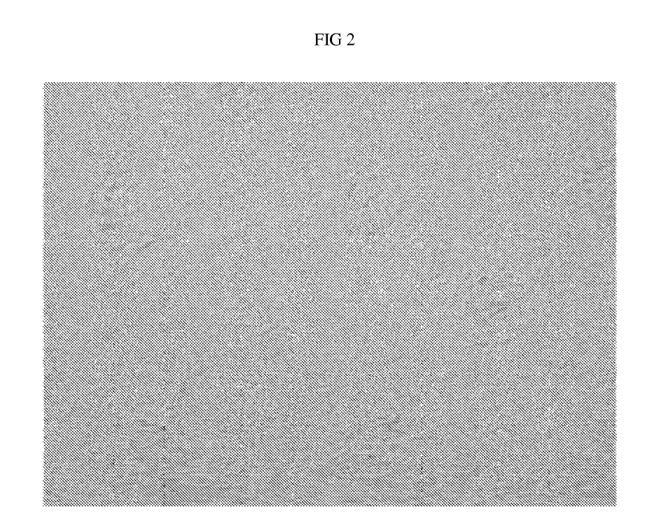

Among all the frozen cell immobilized products prepared in Example 1, frozen cell immobilized products preserved at −80° C. were subjected to a transportation test. Frozen cell immobilized products cryopreserved for two months, one month, or one week were tested. Each product was placed in a polystyrene foam container charged with dry ice, and spaces between the product and the container were stuffed with crushed dry ice, followed by sealing and packaging of the entire container. The container was transported by means of frozen parcel door-to-door overnight delivery service. Thereafter, the container was opened, and the product was preserved at −80° C. for one week. After completion of cryopreservation, an ordinary culture medium (1 mL) was added to the product, and the product was allowed to stand still in an incubator at 37° C. for 10 minutes, followed by replacement of the culture medium with an ordinary 10% (V/V) fetal bovine serum-containing Dulbecco's modified Eagle's medium. Culturing was performed in the thus-replaced medium for 24 hours, and then the shape of cells was observed under a microscope. FIG. 2 shows the results obtained through microscopic observation (other conditions are as follows: water-soluble photosensitive material: material mainly containing polyethylene glycol; substrate: patterned glass thin plate for culture; animal-derived adherent cells: bovine vascular endothelial cells; preculture: 24 hours; freezing culture medium: 10% (V/V) fetal bovine serum-containing Dulbecco's modified Eagle's medium to which 10% (V/V) dimethyl sulfoxide had been added; and cryopreservation period: one month).

As a result, neither removal of cells nor change in shape of cells was observed, and the product was found to be in the same state as before freezing. Subsequently, the product was washed with phosphate buffer, and then cells were recovered from the product by use of a trypsin solution, followed by trypan blue staining, to thereby determine the survival rate of the cells. As a result, the survival rate was found to be 99% or higher under all the experimental conditions.

### Example 4

### Production of Primary Hepatocyte Culture Tool and Performance Evaluation of the Tool

Among the frozen cell immobilized products prepared in Example 1, there was employed a frozen cell immobilized product prepared under the following conditions: water-soluble photosensitive material: material mainly containing polyethylene glycol; substrate: patterned glass thin plate for culture; animal-derived adherent cells: vascular endothelial cells; preculture: 24 hours; freezing culture medium: Cellbanker; and cryopreservation period: one week. The performance of a primary hepatocyte culture tool produced by use of the product was evaluated. In a manner similar to that described in Example 2, thawing and culture medium replacement were carried out, and then culturing was performed in the thus-replaced medium. Two, four, six, or 24 hours after initiation of culturing, the culture medium was removed from the product, and a separately prepared cell suspension of rat primary hepatocytes (culture medium: Williams' E medium, cell concentration: 3.5×10⁵ cells/mL) was seeded onto the product (1 mL/well). One day or three days after initiation of culturing of primary hepatocytes, 150 μM (═μmol/L) testosterone was added to the culture system, and one hour thereafter, the amount of 6β-hydroxytestosterone (i.e., a testosterone metabolite) present in the culture medium was determined through HPLC (high performance liquid chromatography). For a control, in a manner similar to that described above, an experiment was performed on a hepatocyte suspension immediately after preparation thereof. Also, a similar experiment was carried out on a patterned substrate on which hepatocytes had been seeded without freezing of vascular endothelial cells. The results are shown in Table 3.

As shown in Table 3, hepatocytes cultured on the frozen cell immobilized product maintained metabolic activity equal to or higher than that of hepatocytes seeded on the substrate which did not undergo freezing, although the cultured hepatocytes exhibited metabolic activity slightly lower than that of hepatocytes as determined immediately after collection.

TABLE 3

| Number of days of culture | One day (μM) | Three days (μM) |
|---|---|---|
| Seeding of hepatocytes 2 hours | 8.0 | 6.6 |
| after thawing | | |
| Seeding of hepatocytes 24 hours | 7.9 | 5.9 |
| after thawing | | |
| Seeding of hepatocytes without | 6.0 | 7.2 |
| freezing | | |
| Hepatocyte suspension | 10.0 (day 0) | — |

1. A frozen cell immobilized product, characterized in that the cell product comprises a culture substrate having a cell-adhesion region defined in a pattern; animal-derived adherent cells cultured on the cell-adhesion region; and a freezing culture medium; and that the cultured cells are frozen together with the freezing culture medium.

2. A primary hepatocyte culture tool, characterized in that the tool is produced by thawing the frozen cell immobilized product as recited in claim 1, replacing the freezing culture medium with an ordinary culture medium, and seeding primary hepatocytes on the product.

3. A primary hepatocyte culture tool according to the claim 2, wherein thawing is carried out at 30 to 48° C.

4. A method for producing a primary hepatocyte culture tool, characterized in that the method comprises thawing the frozen cell immobilized product as recited in claim 1; replacing the freezing culture medium with an ordinary culture medium; and seeding primary hepatocytes on the product.

5. A method for producing a primary hepatocyte culture tool according to claim 4, wherein thawing is carried out at 30 to 48° C.

6. A method for producing a primary hepatocyte culture tool according to claim 4, wherein seeding of primary hepatocytes is carried out within 24 hours after thawing.

7. A method for producing a primary hepatocyte culture tool according to claim 4, wherein seeding of primary hepatocytes is carried out within three hours after thawing.

8. A method for producing a primary hepatocyte culture tool according to claim 5, wherein seeding of primary hepatocytes is carried out within 24 hours after thawing.

9. A method for producing a primary hepatocyte culture tool according to claim 5, wherein seeding of primary hepatocytes is carried out within three hours after thawing.
